(19) United States
(12) Patent Application Publication
(10) Pub. No.: US 2012/0226240 A1
(43) Pub. Date: Sep. 6, 2012

(54) SYRINGE

(75) Inventors: Tony Bedford, Cambridge (GB); Duncan Bishop, Cambridgeshire (GB); Andrew Gow, Cambridge (GB); Dave Harris, Cambridge (GB); Nick Hawson, Cambridgeshire (GB); Dominic Lloyd-Lucas, Cambridge (GB); Martin Murphy, Cambridgeshire (GB); Christophe Royer, Huningue (FR)

(73) Assignee: NOVARTIS AG, Basel (CH)

(21) Appl. No.: 13/510,213

(22) PCT Filed: Nov. 19, 2010
(86) PCT No.: PCT/EP2010/067869

§ 371 Date: May 16, 2012

(30) Foreign Application Priority Data
Nov. 20, 2009 (EP) 09176607.1

(51) Int. Cl. A61M 5/315

(52) U.S. Cl. 604/211

(57) ABSTRACT

A syringe (2) for dispensing a fluid, the syringe including a barrel (4) including a discharge end (10) defining a discharge passage, and a plunger (15) disposed within the barrel, the plunger being adapted to move within the barrel such that the plunger and discharge end define a variable volume chamber within the barrel and the plunger is capable of displacing fluid from the chamber through the discharge passage; wherein the syringe includes a rotational element (20a, 20b, 24a, 24b) adapted to rotate about longitudinal axis of the syringe and which has a first axial orientation relative to the discharge end of the barrel and a first stop is provided on the plunger and/or the barrel which is adapted to limit axial movement of the plunger in the first orientation of the rotational element, and the rotational element has a second axial orientation relative to the discharge end of the barrel in which the first stop is disengaged and movement of the plunger is permitted to a second stop.

The present invention relates to a syringe and, in particular, to a syringe adapted to permit priming, followed by dosing of a pre-determined dose.

It is often desirable to deliver a relatively small volume of fluid from a syringe in a method that is both accurate and reproducible. Typically, syringes containing fluids are first primed, to ensure that no air or other gas is present in the syringe and then a second, delivery (dosage) step is performed to deliver the required volume of fluid. However, it is difficult to deliver accurately a relatively small volume of fluid during the delivery step using a conventional syringe.

WO01/62319, U.S. Pat. No. 3,934,586 and WO03/004080 all describe dual stage syringes, but none of them addresses the issue of delivering accurately and reproducibly a relatively small volume of a fluid.

According to a first aspect of the present invention, there is provided a syringe for dispensing a fluid, the syringe including a barrel including a discharge end defining a discharge passage, and a plunger disposed within the barrel, the plunger being adapted to move within the barrel such that the plunger and discharge end define a variable volume chamber within the barrel and the plunger is capable of displacing fluid from the chamber through the discharge passage; wherein the syringe includes a rotational element adapted to rotate about longitudinal axis of the syringe and which has a first axial orientation relative to the discharge end of the barrel and a first stop is provided on the plunger and/or the barrel which is adapted to limit axial movement of the plunger in the first orientation of the rotational element, and the rotational element has a second axial orientation relative to the discharge end of the barrel in which the first stop is disengaged and movement of the plunger is permitted to a second stop.

In accordance with the invention, the plunger can be displaced towards the discharge end in the first axial orientation until the first stop is engaged. This constitutes the priming step. The rotational element is then rotated from the first axial orientation to the second axial orientation, whereupon the first stop is disengaged or by-passed and the plunger can be further displaced towards the second stop. The spacing between the first and second stops defines the volume dispensed from the syringe during the delivery step for a specific barrel internal diameter. For relatively small volumes of fluid, the displacement from the first stop to the second stop is relatively short, but accurately controlled by the syringe according to the invention.

The rotational element is adapted to be rotated between the first and second axial orientations. Thus, the second axial orientation is typically angularly displaced from the first axial orientation.

In an embodiment of the invention, the plunger comprises the rotational element. However, in an alternative embodiment, a first part of the plunger may be adapted to rotate relative to discharge end of the barrel and/or a second part of the plunger. In a yet further embodiment, a part of the barrel, e.g. an end part of the barrel opposite to the discharge end, may comprise the rotational element and be adapted to rotate relative to the discharge end of the barrel.

In order to prevent unintended movement from the first axial orientation to the second axial orientation, for example before completion of the priming step, in embodiments where the plunger comprises or includes the rotational element, the plunger may include an orientation controlling element which is adapted to prevent rotation of the plunger from the first orientation to the second orientation until the plunger engages the first stop. This ensures that the plunger remains in the first axial orientation until the priming step is complete and the plunger has engaged the first stop.

In an embodiment of the invention as defined anywhere herein, the orientation controlling element includes a spline (a longitudinally extending rib) carried by the plunger which is located in use within a spline channel defined in at least a part of the barrel. For example, the barrel may include a collar located at the opposite end to the discharge channel and the collar defines the spline channel.

The interaction of the spline carried by the plunger with a spline channel prevents unwanted movement, e.g. rotation, of the plunger relative to the barrel from the first axial orientation to the second axial orientation.

In order to permit movement, such as rotation, of the plunger relative to the barrel, the spline may include a notch which is adapted to permit rotation of the plunger relative to the barrel about its longitudinal axis. The notch may be defined within the spline or it may be defined by a part of the plunger between one end of the spline and an adjacent end of the plunger. In other words, the spline may not extend to the end of the plunger, but have one end spaced from the end of the plunger such that the gap defined between the end of the plunger and the end of the spline defines the notch. In one embodiment the notch, or each notch is more than one is included, is defined so as to allow rotation of the plunger only when the plunger engages the first stop.

The skilled person will appreciate that the plunger may include more than one spline, each of which includes a respective notch such that all of the notches are equally spaced from one end of the plunger. In this arrangement, the notches are circumferentially spaced about the plunger and permit rotation of the plunger when the notches are concentrically arranged with the spline channel. The collar may be removable from the barrel and adapted to engage an end of the barrel.

In such an embodiment, the barrel typically includes corresponding spline channels for each spline.

A common arrangement for plungers is to include a cruciform-shaped shaft. Thus, in an embodiment of the invention as defined anywhere herein, the plunger includes a piston located within the barrel, an elongate shaft extending from the piston and a push button at the end of the shaft opposite to the piston, wherein the plunger shaft has a cruciform cross sectional configuration and each arm of the cruciform shaft defines a notch such that all of the notches are equally spaced from the push button.

In such an embodiment, the spline channel may include a complimentary cruciform shape.

In a further embodiment of the invention, the barrel includes a collar at the opposite end to the discharge end, and one of the collar and the plunger includes an axial projection and the other of the collar and the plunger includes a first stop surface and a recess sized and configured to receive therein the projection, wherein the projection is adapted to align axially with the first stop surface in the first orientation of the plunger and the projection is adapted to align axially with the recess in the second axial orientation, whereby the plunger movement is limited in the first orientation by the interaction of the projection and the first stop surface and further movement of the plunger is permitted in the second orientation by the projection being received into the recess.

In this embodiment, the priming step is limited by the axial projection engaging the first stop surface. The plunger is then moved from the first to the second orientation (e.g. by rotation about its longitudinal axis) in which configuration, the delivery or dosage step can occur. The volume of fluid delivered during the delivery step is proportional to the distance that the projection extends into the recess. [symbol] includes a piston located within the barrel, an elongate shaft extending from the piston and a push button at the end of the shaft opposite to the piston, wherein the push button carries the projection and the collar defines the first stop surface and the recess.

The first stop surface may be defined by a second projection extending from a base surface forming part of either the plunger or the collar. In such an embodiment, the recess may be defined as a portion of the base surface which does not carry the second projection. Thus, the first projection may be axially aligned with the second projection in the first axial orientation and be out of axial alignment with the second projection in the second axial orientation such that, in the second axial orientation, the first projection is capable of sliding past the second projection.

In this embodiment, the maximum displacement of the first projection into the recess is defined by the longest of the first and second projections, where length is defined as the distance by which the relevant projection extends from a respective base surface.

In a further embodiment of the invention, the first projection includes a pair of opposed first projecting elements and the second projection includes a pair of opposed second projecting elements, wherein the first projecting elements are axially aligned with the second projecting elements in the first orientation and the first projecting elements are axially aligned with respective recesses defined between the second projecting elements in the second orientation.

In a still further embodiment, the plunger includes a piston located within the barrel, an elongate shaft extending from the piston and a push button at the end of the shaft opposite to the piston, the push button carrying a pair of opposed first projecting elements extending from a first base surface and defining therebetween a pair of first recesses; and wherein the collar carries a pair of opposed second projecting elements extending from a second base surface and defining therebetween a pair of second recesses; the first projecting elements being axially aligned with the second projecting elements in the first orientation and the first projecting elements being axially aligned with the second recesses and the second projecting elements being axially aligned with the first recesses in the second orientation, whereby interaction of the first and second projecting elements in the first orientation defines the first stop and the interaction of the first projecting elements and/or the second projecting elements with the respective second or first base surface defines the second stop. [symbol] nt of the invention as described anywhere herein, the rotational element may comprise the push button which is adapted to rotate relative to the plunger shaft. Alternatively, the rotational element may comprise the collar, which may be adapted to rotate relative to the barrel and/or the plunger shaft. Further alternatively, the rotational element may comprise a first part of the plunger shaft which is adapted to rotate relative to a second part of the plunger shaft.

In a still further embodiment of the invention as defined anywhere herein, the syringe may include a plurality of second stops. In such an embodiment, the syringe may be a multiple dose syringe, wherein each second stop is axially spaced from its neighbour, such that after the priming step, the syringe may be capable of delivering multiple sequential doses of a medicament by movement of the plunger from one second stop to the next. Additionally or alternatively, the plurality of second stops may permit a dose selection step, wherein the plunger may be axially aligned with a specific one of the second stops to permit dosing with a pre-determined dose. In such an embodiment, each of the second stops may be rotationally displaced from adjacent second stops. In other words, the second stops may be circumferentially spaced relative to each other and each second stop is disposed from the first stop by a different axial distance.

According to a second aspect of the invention, there is provided a plunger assembly for use with a syringe barrel, the assembly including a collar adapted to engage one end of the barrel; and a plunger including a piston adapted to be located within the barrel, an elongate shaft extending from the piston and a push button at the end of the shaft opposite to the piston, wherein the collar is slidably coupled to the elongate shaft, and wherein the plunger has a first axial orientation relative to the collar and a first stop is provided on the plunger and/or the collar which is adapted to limit axial movement of the plunger in the first orientation, and the plunger has a second axial orientation relative to the collar in which the first stop is disengaged and movement of the plunger is permitted to a second stop.

Since the collar of the plunger assembly is adapted to engage one end of the barrel this plunger assembly provides a way of creating a prime and dose syringe as set out above using a standard syringe barrel.

The additional features described and defined herein in connection with the first aspect of the invention may apply equally to the second aspect of the invention. Thus, the second aspect of the invention may include any, some or all of the additional features described hereinabove.

According to a third aspect of the invention, there is provided a syringe kit including a syringe barrel and a plunger assembly according to the second aspect of the invention.

According to a fourth aspect of the invention, there is provided a pre-filled syringe for dispensing a fluid, the syringe including a barrel including a discharge end defining a discharge passage, the barrel containing therein a medicament in fluid form; and a plunger disposed within the barrel, the plunger being adapted to move within the barrel such that the plunger and discharge end define a variable volume chamber within the barrel and the plunger is capable of displacing fluid from the chamber through the discharge passage; wherein the syringe includes a rotational element adapted to rotate about longitudinal axis of the syringe and which has a first axial orientation relative to the discharge end of the barrel and a first stop is provided on the plunger and/or the barrel which is adapted to limit axial movement of the plunger in the first orientation of the rotational element, and the rotational element has a second axial orientation relative to the discharge end of the barrel in which the first stop is disengaged and movement of the plunger is permitted to a second stop.

Another aspect of the invention provides a syringe for dispensing a fluid, the syringe including a barrel including a discharge end defining a discharge passage, and a plunger disposed within the barrel, the plunger being adapted to move within the barrel such that the plunger and discharge end define a variable volume chamber within the barrel and the plunger is capable of displacing fluid from the chamber through the discharge passage; the barrel includes a collar at the opposite end to the discharge end and the syringe includes a rotational element adapted to rotate about longitudinal axis of the syringe and which has a first axial orientation relative to the discharge end of the barrel and a first stop is provided on the plunger and/or the barrel which is adapted to limit axial movement of the plunger in the first orientation of the rotational element, the rotational element has a second axial orientation relative to the discharge end of the barrel in which the first stop is disengaged and movement of the plunger is permitted to a second stop, the plunger includes an orientation controlling element which is adapted to prevent movement of the plunger from the first orientation to the second orientation until the plunger engages the first stop, said orientation controlling element comprising at least one spline carried by the plunger which is located in use within a spline channel defined in at least a part of the barrel, one of the collar and the plunger includes an axial first projection and the other of the collar and the plunger includes a first stop surface and a recess sized and configured to receive therein the projection, wherein the projection is adapted to align axially with the first stop surface in the first orientation of the plunger and the projection is adapted to align [symbol] the second axial orientation, whereby the plunger movement is limited in the first orientation by the interaction of the projection and the first stop surface and further movement of the plunger is permitted in the second orientation by the projection being received into the recess.

The additional features described and defined herein in connection with the first aspect of the invention may apply equally to the fourth aspect of the invention. Thus, the fourth aspect of the invention may include any, some or all of the additional features described hereinabove.

The skilled person will appreciate that the features specified above in connection with embodiments of the invention may be combined with each other and any of the aspects of the invention as defined. Thus, the present invention includes within its scope an aspect of the invention combined with two or more of the features described anywhere herein as optional features. All such combinations of features described herein are considered to be made available to the skilled person.

An Embodiment of the invention will now be described in detail, by way of example only, with reference to the accompanying drawings in which:

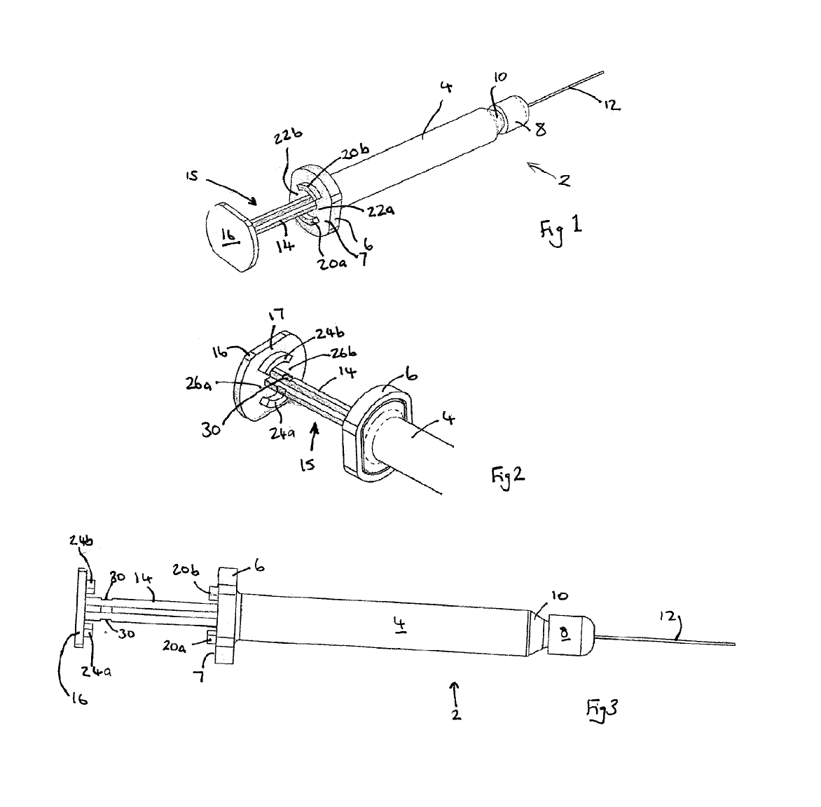

FIG. 1 is a perspective view of a syringe according to the invention showing the collar;

FIG. 2 is a perspective view of the syringe of FIG. 1, showing the push button in detail; and

FIG. 3 is a side elevational view of the syringe of FIGS. 1 and 2.

For the avoidance of doubt, the skilled person will appreciate that in this specification, the terms “up”, “down”, “front”, “rear”, “upper”, “lower”, “width”, etc. refer to the orientation of the components as found in the syringe when installed for normal use as shown in the Figures.

A syringe 2 according to the invention is shown in FIGS. 1, 2 and 3. The syringe 2 includes a barrel body 4 defining a cylinder therein. Located at the rear of the barrel 4 is a collar 6 and at the opposite end, i.e. the front, of the barrel 4 is a discharge end 10 which defines therein a discharge passage. Secured to the discharge end 10 of the barrel 4 is a connecting collar 8 of a hypodermic needle 12.

The skilled person will appreciate that the hypodermic needle connecting collar 8 may be a simple friction fit with the discharge end 10 of the barrel, or it may be adhered to the discharge end 10 of [symbol] locking collar, such as a Luer-Lok collar. The connection of hypodermic needles to syringe barrels is well known in the art and will not be described in detail herein.

A plunger 15 is slidably coupled to the collar 6 and includes a piston (not shown) within the barrel cylinder and an elongate shaft 14 of cruciform cross section which terminates in a flange 16 at the opposite end of the shaft 14 to the piston, such that the rearward facing surface of the flange 16 forms a push button for the plunger 15.

The rearward-facing surface of the collar 6 defines a substantially planar collar base surface 7 from which projects axially a pair of opposed arcuate projections 20a, 20b.

The two opposed projections 20a, 20b prescribe two opposed arcs of a notional circle formed concentrically about the longitudinal axis of the barrel and define therebetween a pair of opposed arcuate gaps 22a, 22b, which also are in the form of opposed arcs of the notional circle, such that the projections 20a, 20b and the gaps 22a, 22b together prescribe the circumference of the notional circle. The arcs prescribed by the projections 20a, 20b are shorter than the arcs defined by the gaps 22a, 22b between the projections 20a, 20b.

FIG. 2 shows a detailed view of the forward facing parts of the flange 16 and of the shaft 14.

The flange 16 defines a substantially planar forward facing base surface 17 from which projects axially a pair of opposed arcuate projections 24a, 24b. The flange projections 24a, 24b are arranged in a substantially identical arrangement to the collar projections 20a, 20b. Thus the flange projections 24a, 24b define therebetween arcuate gaps 26a, 26b and together the flange projections 24a, 24b and the gaps 26a, 26b defined therebetween prescribe the circumference of a notional circle arranged concentrically about the longitudinal axis of the shaft 14.

As with the arrangement of projections 20a, 20b and gaps 22a, 22b on the collar 6, the arcs prescribed by the projections 24a, 24b from the base surface 17 of the flange 16 are shorter than the arcs defined by the gaps 26a, 26b between the projections 24a, 24b.

When the piston is spaced from the discharge end 10 and consequently, the flange 16 is spaced from the collar 6, as shown in the Figures, the collar projections 20a, 20b are spaced apart from the flange [symbol] en spaced apart in this way, the collar projections 20a, 20b are axially aligned with the flange projections 24a, 24b.

The cruciform shaft 14 is slidably located within a complimentary shaped (i.e. cruciform-shaped) though-hole formed in the collar 6. This ensures that the shaft 14 and the flange 16 is maintained in a constant orientation relative to the collar and the barrel when the two pairs of projections 20a, 20b, 24a, 24b are spaced apart and axially aligned.

Each arm of the shaft 14 includes a notch 30 such that all four notches 30 are equally spaced from the base surface 17 of the flange 16. The notches are configured such that they overlap with the body of the collar 6 when the collar projections 20a, 20b contact the flange projections 24a, 24b. When the notches 30 overlap with the body of the collar 6, the shaft 14 is disengaged from the collar 6 and is permitted to rotate about its longitudinal axis relative to the collar 6.

A rotation of 90° about its longitudinal axis re-aligns the cruciform shaft 14 with the cruciform-shaped through-hole through the collar 6. In addition, the collar projections 20a, 20b become axially aligned with the flange gaps 26a, 26b and the flange projections 24a, 24b become axially aligned with the collar gaps 22a, 22b. As the gaps 22a, 22b, 26a, 26b are bigger than the corresponding projections 20a, 20b, 24a, 24b, the projections 20a, 20b, 24a, 24b are capable of sliding past each other axially in this configuration.

In use, the cylinder within the barrel 4 is filled with a fluid, e.g. a medicament in a liquid formulation, by drawing the flange 16 and hence the piston rearwards when the projections 20a, 20b, 24a, 24b are axially aligned.

The syringe is then primed by pressing the push button and axially sliding the flange 16, the shaft 14 and the piston forwards towards the discharge end 10 of the barrel 4. This has the effect of dispelling all air from the needle 12 and the cylinder of the barrel 4.

The priming step is continued until the flange projections 24a, 24b contact the collar projections 20a, 20b, at which point, the notches 30 overlap with the body of the collar 6. The flange is then rotated through 90° whereupon the projections 20a, 20b, 24a, 24b become axially aligned with the corresponding gaps 22a, 22b, 26a, 26b. The push button is then urged forwards once more to deliver the pre-determined dose of the fluid. This delivery step is continued until the flange projections 24a, [symbol] se surface 7 and/or the collar projections 20a, 20b contact the flange base surface 17.

The volume of the dose delivered during the delivery step is calculated by multiplying the cross sectional area of the cylinder of the barrel by the length of the longest of the projections 20a, 20b, 24a, 24b, where the length of the projections is defined as the distance the relevant projection extends from the respective base surface. Thus, the delivered volume can be varied either by varying the cross-sectional area of the cylinder of the barrel 4 or by varying the length of the projections or both.

Tests of the syringe described above were carried out as follows:

### Test Apparatus

The syringe plunger and collar were assembled to a sample 1 ml syringe barrel. A needle was fitted to the discharge end of the syringe barrel.

### Preparation and Measurement

The syringe was filled with a nominal volume of RO/DI water, held vertically and tapped to release air bubbles, and primed to the first stop. A clean Eppendorf Tube was tared on an analytical balance. The plunger was rotated from the first axial orientation to the second axial orientation to permit dosing, and the plunger depressed to the second stop, discharging the dose into the vial.

The vial was then closed and weighed.

The test was repeated, re-using the syringe, but taking a clean vial.

### Calculation

Dose mass was calculated from the difference in weight of the vial.
Dose volume was calculated from the mass by dividing by density.
Density=998.022 kg/m³ at lab conditions of 21° C. and 42% RH

### Apparatus

Sartorius ME235S-OCE analytical balance (5 decimal places/1g)

### 300 μl Eppendorf Tubes

### Syringe Device

TABLE 1

| | mean | Standard deviation | lowest | highest |
|---|---|---|---|---|
| Mass (g) | 0.0445 | 0.0024 | 0.0407 | 0.0483 |
| Volume (μl) | 44.4 | 2.4 | 40.8 | 48.4 |

The results from N = 10 measurements

The dose delivered by the prototype in the tests is in the range 44.4±3.8 μl i.e. 40.8 to 48.4 μl.

The syringe delivered a well controlled dose, within a tolerance of ±3.8 μl, which is within the tolerance of ±7.5 μl associated with the acceptance limits.

1. A syringe for dispensing a fluid, the syringe including a barrel including a discharge end defining a discharge passage, and a plunger disposed within the barrel, the plunger being adapted to move within the barrel such that the plunger and discharge end define a variable volume chamber within the barrel and the plunger is capable of displacing fluid from the chamber through the discharge passage, the syringe including a rotational element adapted to rotate about longitudinal axis of the syringe and which has a first axial orientation relative to the discharge end of the barrel and a first stop is provided on the plunger and/or the barrel which is adapted to limit axial movement of the plunger in the first orientation of the rotational element, and the rotational element has a second axial orientation relative to the discharge end of the barrel in which the first stop is disengaged and movement of the plunger is permitted to a second stop, wherein the plunger is the rotational element and is adapted to be rotated between the first and second axial orientations and the plunger includes an orientation controlling element which is adapted to prevent movement of the plunger from the first orientation to the second orientation until the plunger engages the first stop.

2. (canceled)

3. (canceled)

4. A syringe according to claim 1, wherein the orientation controlling element includes a spline carried by the plunger which is located in use within a spline channel defined in at least a part of the barrel.

5. A syringe according to claim 4, wherein the barrel includes a collar at the opposite end to the discharge end and the collar defines the spline channel.

6. A syringe according to claim 4, wherein the spline defines a notch which is adapted to permit rotation of the plunger relative to the barrel about its longitudinal axis.

7. A syringe according to claim 4, wherein the plunger includes a plurality of splines and each spline defines a notch such that the notches are equally spaced from one end of the plunger.

8. A syringe according to claim 5, wherein the plunger includes a piston located within the barrel, an elongate shaft extending from the piston and a push button at the end of the shaft opposite to the piston, wherein the plunger shaft has a cruciform cross sectional configuration and each arm of the cruciform shaft defines a notch such that all of the notches are equally spaced from the push button.

9. A syringe according to claim 1, wherein the barrel includes a collar at the opposite end to the discharge end and one of the collar and the plunger includes an axial projection and the other of the collar and the plunger includes a first stop surface and a recess sized and configured to receive therein the projection, wherein the projection is adapted to align axially with the first stop surface in the first orientation of the plunger and the projection is adapted to align axially with the recess in the second axial orientation, whereby the plunger movement is limited in the first orientation by the interaction of the projection and the first stop surface and further movement of the plunger is permitted in the second orientation by the projection being received into the recess.

10. A syringe according to claim 7, wherein the plunger includes a piston located within the barrel, an elongate shaft extending from the piston and a push button at the end of the shaft opposite to the piston, and wherein the push button carries the projection and the collar defines the first stop surface and the recess.

11. A syringe according to claim 7, wherein the first stop surface is defined by a second projection extending from a base surface and the recess is defined by a portion of the base surface which does not carry the second projection.

12. A syringe according to claim 9, wherein the first projection includes a pair of opposed first projecting elements and the second projection includes a pair of opposed second projecting elements, wherein the first projecting elements are axially aligned with the second projecting elements in the first orientation and the first projecting elements are axially aligned with respective recesses defined between the second projecting elements in the second orientation.

13. A syringe according to claim 10, wherein the plunger includes a piston located within the barrel, an elongate shaft extending from the piston and a push button at the end of the shaft opposite to the piston, the push button carrying a pair of opposed first projecting elements extending from a first base surface and defining therebetween a pair of first recesses; and wherein the collar carries a pair of opposed second projecting elements extending from a second base surface and defining therebetween a pair of second recesses; the first projecting elements being axially aligned with the second projecting elements in the first orientation and the first projecting elements being axially aligned with the second recesses and the second projecting elements being axially aligned with the first recesses in the second orientation, whereby interaction of the first and second projecting elements in the first orientation defines the first stop and the interaction of the first projecting elements and/or the second projecting elements with the respective second or first base surface defines the second stop.

14. A syringe as claimed in claim 1, in which the barrel includes a collar at the opposite end to the discharge end and the plunger includes an orientation controlling element which is adapted to prevent movement of the plunger from the first orientation to the second orientation until the plunger engages the first stop, said orientation controlling element comprising at least one spline carried by the plunger which is located in use within a spline channel defined in at least a part of the barrel, one of the collar and the plunger includes an axial first projection and the other of the collar and the plunger includes a first stop surface and a recess sized and configured to receive therein the projection, wherein the projection is adapted to align axially with the first stop surface in the first orientation of the plunger and the projection is adapted to align axially with the recess in the second axial orientation, whereby the plunger movement is limited in the first orientation by the interaction of the projection and the first stop surface and further movement of the plunger is permitted in the second orientation by the projection being received into the recess.

15. A plunger assembly for use with a syringe barrel, the assembly including a collar adapted to engage one end of the barrel; and a plunger including a piston adapted to be located within the barrel, an elongate shaft extending from the piston and a push button at the end of the shaft opposite to the piston, wherein the collar is slidably coupled to the elongate shaft, and wherein the plunger has a first axial orientation relative to the collar and a first stop is provided on the plunger and/or the collar which is adapted to limit axial movement of the plunger in the first orientation, and the plunger has a second axial orientation relative to the collar in which the first stop is disengaged and movement of the plunger is permitted to a second stop and the plunger includes an orientation controlling element which is adapted to prevent movement of the plunger from the first orientation to the second orientation until the plunger engages the first stop.
